(19) United States
(12) Patent Application Publication
(10) Pub. No.: US 2025/0367478 A1
(43) Pub. Date: Dec. 4, 2025

(54) HISTOTRIPSY THERAPY SYSTEMS AND METHODS FOR THE TREATMENT OF BRAIN TISSUE

(71) Applicant: THE REGENTS OF THE UNIVERSITY OF MICHIGAN, Ann Arbor, MI (US)

(72) Inventors: Zhen XU, Ann Arbor, MI (US); Jonathan SUKOVICH, Ann Arbor, MI (US); Aditya S. PANDEY, Ann Arbor, MI (US); Charles A. CAIN, Ann Arbor, MI (US); Hitinder S. GURM, Ann Arbor, MI (US)

(21) Appl. No.: 19/006,948

(22) Filed: Dec. 31, 2024

Related U.S. Application Data
(63) Continuation of application No. 17/407,780, filed on Aug. 20, 2021, now Pat. No. 12,220,602.
Application No. 17/407,780 is a continuation of application No. 15/737,761, filed on Dec. 19, 2017, now Pat. No. 11,135,454.
(60) Provisional application No. 62/184,179, filed on Jun. 24, 2015.

(51) Int. Cl. A61N 7/02; A61N 7/00

(52) U.S. Cl. CPC A61N 7/02; A61N 7/00; A61N 2007/003; A61N 2007/0039; A61N 2007/0047; A61N 2007/0065; A61N 2007/0078; A61N 2007/0086; A61N 2007/0095

(57) ABSTRACT

A histotripsy therapy system configured for the treatment of brain tissue is provided, which may include any number of features. In one embodiment, the system includes an ultrasound therapy transducer, a drainage catheter, and a plurality of piezoelectric sensors disposed in the drainage catheter. The ultrasound therapy is configured to transmit ultrasound pulses into the brain to generate cavitation that liquefies a target tissue in the brain. The drainage catheter is configured to detect the ultrasound pulses. An aberration correction algorithm can be executed by the system based on the ultrasound pulses measured by the drainage catheter to automatically correct for an aberration effect caused by the ultrasound pulses passing through a skullcap of the patient.

### CROSS REFERENCE TO RELATED APPLICATIONS

This application is a continuation of U.S. patent application Ser. No. 17/407,780, filed Aug. 20, 2021, which is a continuation of U.S. patent application Ser. No. 15/737,761, filed Dec. 19, 2017, now U.S. Pat. No. 11,135,454, which is the national phase entry of International Application No. PCT/US2016/039020, filed Jun. 23, 2016, which claims the benefit of U.S. Provisional Patent Application No. 62/184,179, filed Jun. 24, 2015, titled “HISTOTRIPSY THERAPY SYSTEMS AND METHODS FOR THE TREATMENT OF INTRACEREBRAL HEMORRHAGE”, all of which are incorporated by reference in their entirety.

### GOVERNMENT LICENSE RIGHTS

This invention was made with government support under Grant Number NS093121 awarded by the National Institute of Health. The government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to treating tissue with cavitation created by ultrasound therapy. More specifically, this disclosure relates to treatment of brain tissue or disorders of the brain, such as intracerebral hemorrhage (ICH) or brain tumors, with ultrasound therapy.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) bubble clouds appear bright on ultrasound imaging thereby confirming correct targeting and localization of treatment; 3) treated tissue appears darker (hypoechoic) on ultrasound imaging, so that the operator knows what has been treated; and 4) Histotripsy produces lesions in a controlled and precise manner. It is important to emphasize that unlike microwave, radiofrequency, or high-intensity focused ultrasound (HIFU), Histotripsy is not a thermal modality.

The rupture of blood vessels in the brain can lead to bleeding and clotting (hematoma) inside the brain, termed as hemorrhagic stroke or intracerebral hemorrhage (ICH). ICH accounts for 10-15% of all strokes. Current mainstay treatment remains craniotomy, a highly invasive surgery to remove the clot, associated with severe damage to the brain neurological function.

Minimally invasive (MIS) stereotactic approaches have been investigated to drain the hematoma via a catheter and thrombolytic drug (tPA) over several days. However, there are severe complications associated with tPA, and the functional outcome for ICH survivors is not improved, likely due the long treatment time allowing neurological damage to develop.

Recent preclinical studies show that, using magnetic resonance guided focused ultrasound (MRgFUS) applied outside the skullcap, the clot in the brain can be liquefied without drugs and aspirated out with a needle. However, the MRgFUS treatment time is still not short enough to avoid neurological damage (up to 3 hours for 40 mL clot). It is highly costly due to the long MRI time required, and cannot treat clots within 2 cm distance from the skullcap.

A skullcap in the ultrasound pathway can cause significant attenuation and defocusing (aberration effect) of ultrasound signals passing through the skullcap. For aberration correction, MRgFUS uses a skullcap profile extracted from prior 3D CT scans of the patient brain. However, during MRgFUS treatment, as it is impossible to put the patient in the exact same position as the previous scan, MRI is needed to guide and monitor precise focusing through the skullcap. The process is complex and highly costly.

Furthermore, all the current methods are not effective for large hematoma (>40 mL). There is a clear unmet need for a better ICH therapy that can minimally invasively and rapidly reduce the hematoma in the brain without tPA, which will allow the ICH patients to recover without significant neurological damage.

### SUMMARY OF THE DISCLOSURE

Histotripsy produces tissue fractionation through dense energetic bubble clouds generated by short, high-pressure, ultrasound pulses. When using pulses shorter than 2 cycles, the generation of these energetic bubble clouds only depends on where the peak negative pressure (P−) exceeds an intrinsic threshold for inducing cavitation in a medium (typically 26-30 MPa in soft tissue with high water content).

A method of transmitting ultrasound energy into a brain of a human patient is provided, comprising the steps of placing a drainage catheter within a target tissue in the brain of the human patient, positioning a focus of a plurality of transducer elements of a therapy transducer within the target tissue, transmitting ultrasound pulses from each of the plurality of transducer elements into the target tissue, detecting the ultrasound pulses with one or more piezoelectric sensors positioned on or in the drainage catheter, and adjusting the transmission of ultrasound pulses from the plurality of transducer elements with an aberration correction algorithm based on the detected ultrasound pulses to automatically correct for an aberration effect caused by the ultrasound pulses passing through a skullcap of the human patient.

In one embodiment, the target tissue comprises a clot or hemorrhage. In another embodiment, the target tissue comprises a brain tumor.

In another embodiment, the method further comprises forming a bubble cloud on the target tissue with the ultrasound pulses.

In some embodiments, the method further comprises liquefying the target tissue with the ultrasound pulses.

In another embodiment, the method comprises draining the liquefied target tissue from the brain with the drainage catheter.

In one embodiment, adjusting the transmission of ultrasound pulses from the plurality of transducer elements with the aberration correction algorithm based on the detected ultrasound pulses further comprises determining a propagation time for the ultrasound pulses to travel from each of the plurality of transducer elements of the therapy transducer to the one or more piezoelectric sensors, calculating a time delay of the propagation time between each of the plurality of transducer elements and a reference element of the therapy transducer, and adjusting the transmission of ultrasound pulses from the plurality of transducer elements based on the calculated time delays.

In one embodiment, the one or more piezoelectric sensors comprises first and second piezoelectric sensors. In this embodiment, adjusting the transmission of ultrasound pulses from the plurality of transducer elements with the aberration correction algorithm based on the detected ultrasound pulses further comprises determining a propagation time for the ultrasound pulses to travel from each of a plurality of transducer elements of the therapy transducer to the first and second piezoelectric sensors, calculating a distance between the first and second piezoelectric sensors using projections of the first and second piezoelectric sensors onto a ray from each of the plurality of transducer elements to a midpoint of the first and second piezoelectric sensors, calculating a travel direction and a time of travel of the ultrasound pulses from each of the plurality of transducer elements to the midpoint of the first and second piezoelectric sensors, calculating a stand-off distance between the focus and the midpoint for each of the plurality of transducer elements, and calculating a time delay of each of the plurality of transducer elements based on the distance between the first and second piezoelectric sensors, the midpoint, and the stand-off distance.

In one embodiment, the method comprises placing the one or more piezoelectric sensors within or adjacent to the focus. In another embodiment, the placing step further comprises advancing the drainage catheter through a hole of the therapy transducer.

In another embodiment, the method comprises electronically or mechanically steering the focus to fully liquefy the target tissue.

An ultrasound system configured to treat a target tissue in a brain of a human patient is also provided, comprising a pulse generator and an amplifier, an ultrasound therapy transducer coupled to the pulse generator and the amplifier and having a plurality of transducer elements configured to transmit ultrasound pulses through a skullcap of the human patient towards a focal point within the target tissue in the brain to generate cavitation, a drainage catheter comprising one or more piezoelectric sensors, the drainage catheter adapted to be placed within the brain near the focal point to measure the ultrasound pulses, an electronic controller coupled to the pulse generator, the amplifier, the ultrasound therapy transducer, and the piezoelectric sensors of the drainage catheter, the electronic controller being configured to control transmission of the ultrasound pulses and adjust the transmission of ultrasound pulses from each of the plurality of transducer elements by executing an aberration correction algorithm based on the ultrasound pulses detected by the drainage catheter to automatically correct for an aberration effect caused by the ultrasound pulses passing through the skullcap of the human patient.

In one embodiment, the ultrasound therapy transducer is configured to transmit histotripsy therapy pulses to generate cavitation to liquefy the target tissue within the brain of the human patient.

In another embodiment, the drainage catheter includes drainage ports configured to drain the liquefied target tissue from the human patient.

In some embodiments, the one or more piezoelectric sensors comprises exactly one piezoelectric sensor. In this embodiment, the aberration correction algorithm comprises determining a propagation time for the ultrasound pulses to travel from each of the plurality of transducer elements of the therapy transducer to piezoelectric sensor, calculating a time delay of the propagation time between each of the plurality of transducer elements and a reference element of the therapy transducer, and adjusting the transmission of ultrasound pulses from the plurality of transducer elements based on the calculated time delays.

In some embodiments, the one or more piezoelectric sensors comprises first and second piezoelectric sensors. In this embodiment the aberration correction algorithm comprises determining a propagation time for the ultrasound pulses to travel from each of the plurality of transducer elements of the therapy transducer to the first and second piezoelectric sensors, calculating a distance between the first and second piezoelectric sensors using projections of the first and second piezoelectric sensors onto a ray from each of the plurality of transducer elements to a midpoint of the first and second piezoelectric sensors, calculating a travel direction and a time of travel of the ultrasound pulses from each of the plurality of transducer elements to the midpoint of the first and second piezoelectric sensors, calculating a stand-off distance between the focus and the midpoint for each of the plurality of transducer elements, and calculating a time delay of each of the plurality of transducer elements based on the distance between the first and second piezoelectric sensors, the midpoint, and the stand-off distance.

In one embodiment, the therapy transducer comprises a hole through which the drainage catheter is configured to be advanced into the brain of the human patient.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a Histotripsy therapy system.

FIG. 2 shows schematic of drainage catheter and the guidewire with miniature piezoelectric sensors.

FIG. 3 illustrates the ray tracing algorithm to correct aberration of ultrasound pulses propagating through the skullcap in order to achieve focusing of histotripsy therapy through the skullcap.

FIGS. 4-5 illustrate one embodiment and method for treating brain tissue with Histotripsy ultrasound therapy.

### DETAILED DESCRIPTION

Histotripsy is a noninvasive, cavitation-based therapy that uses very short, high-pressure ultrasound pulses to generate a dense, energetic, lesion-producing bubble cloud. This Histotripsy treatment can create controlled tissue erosion when it is targeted at a fluid-tissue interface and well-demarcated tissue fractionation when it is targeted within bulk tissue. Additionally, Histotripsy has been shown to be capable of fragmenting model kidney stones using surface erosion that is mechanistically distinct from conventional shockwave lithotripsy (SWL). Histotripsy therapy can be guided and monitored using ultrasound B-mode imaging in real-time, since 1) the cavitating bubble cloud appears as a temporally changing hyperechoic region in B-mode imaging, allowing the treatment to be precisely targeted, and 2) the echogenicity of the targeted region decreases as the degree of tissue fractionation increases, which can be used as a way of monitoring lesion production (image feedback) in real-time.

Generally in Histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This has been called the “shock scattering mechanism”. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without these intense shock fronts developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on one or two negative half cycle(s) of the applied ultrasound pulses exceeding an “intrinsic threshold” of the medium (the “intrinsic threshold mechanism”). This threshold can be in the range of 26-30 MPa for soft tissues with high water content, such as tissues in the human body. Using this intrinsic threshold mechanism, the spatial extent of the lesion is well-defined and more predictable.

With peak negative pressures (P−) not significantly higher than this threshold, sub-wavelength reproducible lesions as small as half of the-6 dB beamwidth of a transducer can be generated.

Histotripsy has the potential to overcome the drawbacks of conventional treatment of ICH to provide minimally invasive, rapid reduction of hematoma in the brain, without thrombolytic drugs and regardless the size of the hematoma. Systems and methods described herein transmit microsecond-length ultrasound pulses at high pressures to generate a dense cavitation cloud of microbubbles using pre-existing gas nuclei in the clot within the focal region. The rapid expansion and collapse of the microbubbles induces high strain and stress to adjacent cells to fractionate the cells to liquid-like acellular homogenate.

According to some embodiments, Histotripsy can be used treat brain tissue or disorders of the brain, such as ICH or brain tumors. In one embodiment, Histotripsy can be used to liquefy a clot or a brain tumor through a skullcap of a human patient, and the resulting liquid can then be drained via a drainage catheter, without the use of thrombolytic drugs or external agents. For example, Histotripsy can be used to liquefy in vitro clots of 40 mL through a human skullcap within 30 minutes, which is six-fold faster than MRgFUS. With parameter optimization, the treatment time can be shortened by more than an order of magnitude compared to MRgFUS. These optimized parameters can be used to treat clots larger than 40 mL and at locations within 2 cm to the skullcap. The systems and methods described herein enable rapid clot removal even for clots >40 mL, in a minimally invasive approach, and eliminate the need for thrombolytic drugs and MRI, thereby substantially improving ICH and brain tumor therapy.

According to embodiments described herein that use histotripsy for treating the brain, a catheter can be placed in a target tissue, such as a clot or tumor within the brain of a patient. One or more acoustic hydrophones or PZT sensors can be integrated to a guidewire placed inside the catheter, which can then be inserted into the target tissue in the brain to directly measure ultrasound signals from a histotripsy therapy transducer positioned outside the patient. The timing of pulse transmission from all elements of the histotripsy therapy transducer can be re-aligned to refocus through the skullcap by using the timing of the ultrasound signal received at the sensor from each element of histotripsy therapy transducer. The sensor(s) and associated aberration correction algorithm for transcranial histotripsy therapy described herein is novel and can provide a cost-effective and simplified device to guide and monitor transcranial histotripsy therapy without CT or MRI.

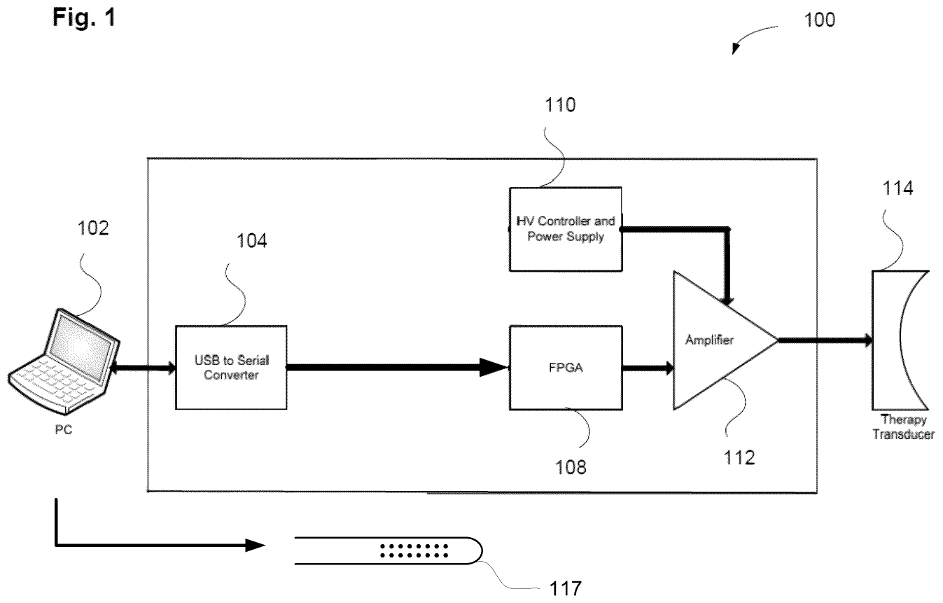

FIG. 1 illustrates a Histotripsy system configured to generate cavitation bubbles or bubble clouds in tissue according to the methods and embodiments described herein. A Histotripsy system and generator is configured to generate complex waveforms in order to support the ultrasound pulse sequences described herein. A simplified block diagram of system 100 is shown in FIG. 1. The main components of the system are: Computer/controller 102, USB to Serial Converter 104, FPGA (Field Programmable Gate Array) 108, High Voltage Controller and Power Supply 110, Amplifier 112, and Therapy Transducer 114, and Drainage Catheter 117.

All controls for the generator can be established using a “Histotripsy Service Tool” software that can run on the computer/controller 102 (e.g., a standard PC, laptop, tablet, or other electronic computing system) and communicates to the generator via a connector such as a wireless, USB, or serial communication 104. The controller 102 can include a non-transitory computer-readable storage medium configured to store a set of instructions capable of being executed by the controller.

The system 100 can be configured to receive multiple sets of different driving parameters and loop them, which give the ability to the user to create wide range of custom sequences where all parameters (pulse repetition frequency (PRF), voltage amplitude, number of cycles, number of pulses per set, frequency, transducer element channels enabled, and time delays) can be set differently for every pulse generated. Time delays between pulses can be specified by the PRF for a parameter set or by specifying them manually/individually on a pulse-by-pulse basis.

For overall voltage amplitude regulation, level of high voltage can be changed accordingly through the HV Controller 110. This method cannot be used for dynamic voltage amplitude changes between two pulses since it will take too long for all capacitors on the HV line to discharge. For dynamic voltage amplitude changes between pulses, PWM (pulse width modulation) can be used at the FPGA 108 where the duty cycle of the capacitor-charging pulse may be modulated in order to produce the desired pulse voltage and resultant pressure amplitude.

### USB to Serial Converter

USB to Serial converter 104 can convert USB combination to serial in order to communicate from the PC or electronic controller to the FPGA. It should be understood that other converters (or none at all) may be used in embodiments where the connection between the generator and the controller is not a USB connection.

### FPGA

The FPGA 108 receives the information from the PC or electronic controller 102 and it can generate the complex pulsing sequence that is required to drive the amplifier 112. The FPGA can run on 100 MHz clock since speed of pulsing is critical to be timed in at least 10 ns increments.

### High Voltage Controller and Power Supply

The High Voltage Controller and Power Supply 110 determines the level of DC voltage that needs to be supplied to the amplifier circuitry in order to have an adequate voltage amplitude level at the output of the amplifier.

### Amplifier

The Amplifier 112 receives pulses generated by the FPGA and is supplied with high voltage from High Voltage Controller and Power Supply. It generates high voltage amplitude pulses that are fed to the Therapy Transducer 114 through the matching network components which properly matches the impedance of the therapy transducer to the impedance of the amplifier. It can be necessary to use a large number of capacitors that can store enough energy to support peak current demand during the generation of high voltage amplitude pulses.

### Therapy Transducer

The Therapy Transducer 114 can be a single element transducer, or a multi-element ultrasound therapy transducer comprising a plurality of transducer elements and configured to generate and deliver the ultrasound therapy pulses described herein into tissue or other mediums. In some embodiments, the multi-element ultrasound therapy transducer can generate ultrasound pulses in two or more frequencies. The active transducer elements of the Therapy Transducer can be piezoelectric transducer elements. In some embodiments, the transducer elements can be mounted to an acoustic lens with a common geometric focus.

In other embodiments, the transducer elements can comprise a phased array that is optimized with steering parameters to maximize treatment speed and locations for transcranial histotripsy clot liquefaction without overheating the skullcap. Overheating the skullcap is the major limitation to restrain the treatment speed and location for transcranial ultrasound therapy. Proposed parameter optimization will ensure a rapid brain tissue treatment and minimize the heating to the skullcap. In some embodiments, the therapy transducer can achieve brain tissue liquefaction rates greater than 1 mL/min, which is orders of magnitude faster than passive thrombolytic action.

The therapy transducer can be configured to generate cavitation through the skullcap with a single ultrasound pulse having one high negative pressure phase lasting approximately 1-4 μs, where the peak negative pressure of the pulse directly exceeds the “intrinsic threshold” for cavitation of the medium (approximately 27 MPa for brain tissue such as clots). The focus of the therapy transducer can be electrically steered to other locations to cover a large treatment volume, and the treatment time can be shortened by more than an order of magnitude compared to other therapy modalities. In some embodiments, the focal steering rate can be kept below 1% duty cycle to avoid overheating the skullcap.

According to the systems and methods described herein, histotripsy brain therapy can be performed without real-time imaging. CT scan may be needed as part of the target tissue diagnosis but is performed prior to the treatment. Using prior CT scan and stereotactic approach, the drainage hydrophone can be placed inside the clot, and the precise position of the catheter tip with regard to the clot position is known. The focus from the histotripsy therapy transducer can then be steered to liquefy a large portion of the brain tissue, leaving a thin rim of the tissue to avoid damage to adjacent brain tissue.

### Drainage Catheter

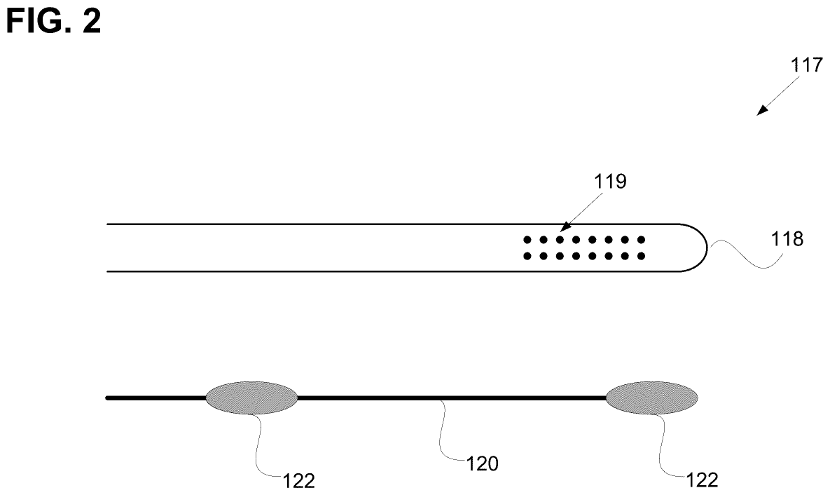

FIG. 2 is an expanded view of the drainage catheter 117 of the system, which can comprise a sheath portion 118 and a guidewire portion 120. The sheath portion can comprise a flexible material and can include one or more drainage ports 119 to facilitate the removal of bodily fluids or tissues through the catheter. The guidewire portion 120 can be insertable into the sheath portion 118 for steering the catheter to the target region in tissue. The drainage catheter can further include one or more piezoelectric (PZT) sensors 122 disposed along the guidewire portion 120. The embodiment of FIG. 2 shows 2 PZT sensors, but it should be understood that any number of PZT sensors can be implemented. For example, some embodiments utilize a single PZT sensor. The PZT sensors of the catheter can be configured to measure ultrasound pulse waveforms from individual elements of the therapy transducer 114 to extract time delays between waveforms transmitted by the therapy transducer. The time delays can then be used by the system for aberration correction.

In addition, the PZT sensors can also be used to monitor the initiation and maintenance of cavitation, which is an indication of successful histotripsy therapy and can be monitored as increased acoustic emission from the cavitation site. As the attenuation caused by the skullcap can vary across patients, such real-time cavitation detection can be used to identify the power needed to initiate cavitation for an individual patient.

Software and hardware can be configured to automatically control the pulse transmission from each element of the therapy transducer sequentially and to collect and store the signals from the PZT sensors. With only a few microseconds necessary to transmit a single pulse from one element at one time and ˜100 μs for the ultrasound to travel from the element to the hydrophone, the entire data acquisition can be accomplished rapidly within a second using the automatic package.

### Histotripsy Service Tool and Electronic Controller

Histotripsy Service Tool is software that can be run on any PC or computing system (e.g., electronic controller) and may be used for controlling the system. The Histotripsy Service Tool can start/stop the therapy with the therapy transducer, set and read the level of high voltage, therapy parameters (PRF, number of cycles, duty ratio, channel enabled and delay, etc.), and set and read other service and maintenance related items. The Histotripsy Service tool and Electronic Controller can be configured to set/read working parameters, start/stop the therapy, etc. It can use internal flash memory or other electronic storage media to store all the parameters. The Histotripsy Service Tool and Electronic Controller can communicate to the FPGA 108 all driving parameters that are necessary to generate complex pulsing. They can also communicate using serial communication or other electronic communication to the high voltage controller and power supply 110 where it can set/read the proper level of driving voltage.

The Histotripsy Service Tool and the Electronic controller can be coupled to the therapy transducer and the PZT sensors of the drainage catheter to use feedback from the drainage catheter during transcranial Histotripsy therapy. When ultrasound pulses propagate through a human skullcap, an aberration effect results in the peak negative pressure of the ultrasound pulses being reduced. In some experiments, the aberration effect of the skullcap has been shown to reduce the peak negative pressure to approximately 20% or less of the free-field condition of the pulses.

In one embodiment, the PZT sensors of the drainage catheter can measure the ultrasound pulse signal from each transducer element of the therapy transducer, and the Histotripsy Service Tool and the Electronic control can use these measurements and execute and aberration correction algorithm to adjust the timing of electrical pulses to each transducer element to correct for the aberration effect. The software and hardware can then automatically control the pulse transmission from each element sequentially and collect and store the measured signals. With only a few microseconds necessary to transmit a single pulse from one element at one time and ˜100 μs for the ultrasound to travel from each element to the PZT sensors, the entire data acquisition can be accomplished within a second using the proposed automatic package.

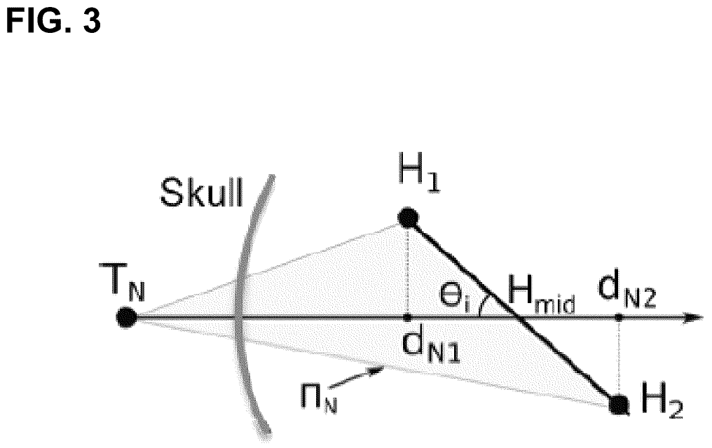

An aberration correction algorithm based on ray-tracing is configured to process the measured signal from the PZT sensors to achieve precise focusing and electrical or mechanical focal steering of the therapy transducer through the skullcap. In the specific embodiment described below, the system can include two or more PZT sensors. The algorithm contains three steps, and is illustrated in FIG. 3. The steps are as follows: 1) Using the known locations of two or more PZT sensors within the catheter (H₁,H₂) and the emitting transducer element (T_{N}) of the therapy transducer, a plane, Π_{N}, is defined onto which the travel direction of the rays are restricted. H₁ and H₂ are assumed to be far enough from TN that the emitted signals from each individual element are effectively traveling as plane waves. The propagation time (t₁ and t₂) for the ultrasound to travel from T_{N} to H₁ and H₂ can be calculated based on the time period between the signal arrival at the PZT sensor and its transmission from the transducer element. Using Δt=t₁−t₂, the distance between d_{N1} and d_{N2} is calculated as dist(d_{N1}−d_{N2})=Cₜᵢₛₛᵤₑ*Δt, where d_{N1} and d_{N2} are the projections of H₁ and H₂ onto the ray from T_{N} to the midpoint of the two sensors, H_{mid}. The travel direction θᵢ and the time of travel of the wave from TN to H_{mid} can then be calculated. 2) Knowing θᵢ, a plane orthogonal to this wave propagation, Πₒᵣₜₕ, can be defined and centered at the H_{mid}. Then assuming plane wave propagation, the requisite time delay of each transducer element can be calculated for a given focal location, fₙ, by calculating the stand-off distance, dₓ, between Πₒᵣₜₕ and fₙ, and plugging into the equation T(fₙ)=t_{mid}+dₓ/Cₜᵢₛₛᵤₑ. 3). Based on the time delay calculated for all steering locations within the treatment volume, a steering pattern can be generated in the software. The software can be configured to control steering parameters as well as cavitation monitoring, and can incorporate the aberration correction algorithm to automatically collect and process the PZT sensor signals and generate adjusted steering patterns.

In one embodiment, and aberration correction algorithm based on time delays is used to achieve precise focusing and electrical or mechanical focal steering of the therapy transducer through the skullcap. In the specific embodiment described immediately below, a single PZT sensor can be used. According to this embodiment, the algorithm comprises determining a propagation time for the ultrasound pulses to travel from each of the plurality of transducer elements of the therapy transducer to the piezoelectric sensor, calculating a time delay of the propagation time between each of the plurality of transducer elements and a reference element of the therapy transducer, and adjusting the transmission of ultrasound pulses from the plurality of transducer elements based on the calculated time delays.

One limitation of ultrasound transcranial therapy is overheating to the skullcap. To address this issue, a number of strategies may be employed in addition to parameter optimization. The order in which certain elements are fired can be alternated to reduce the local heating caused by individual elements. Heat may also be reduced by using cold water as the ultrasound coupling medium to the skullcap.

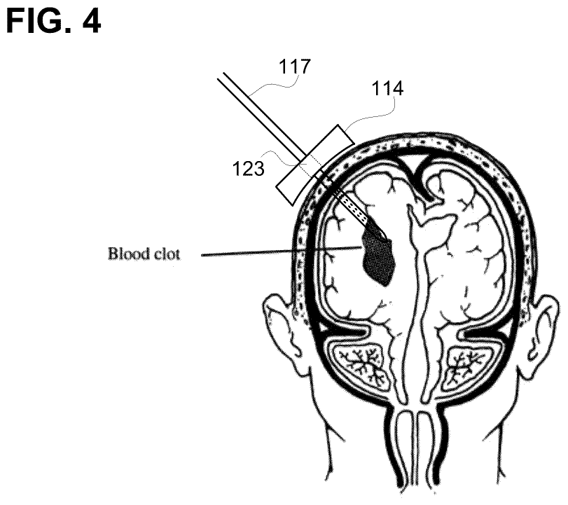

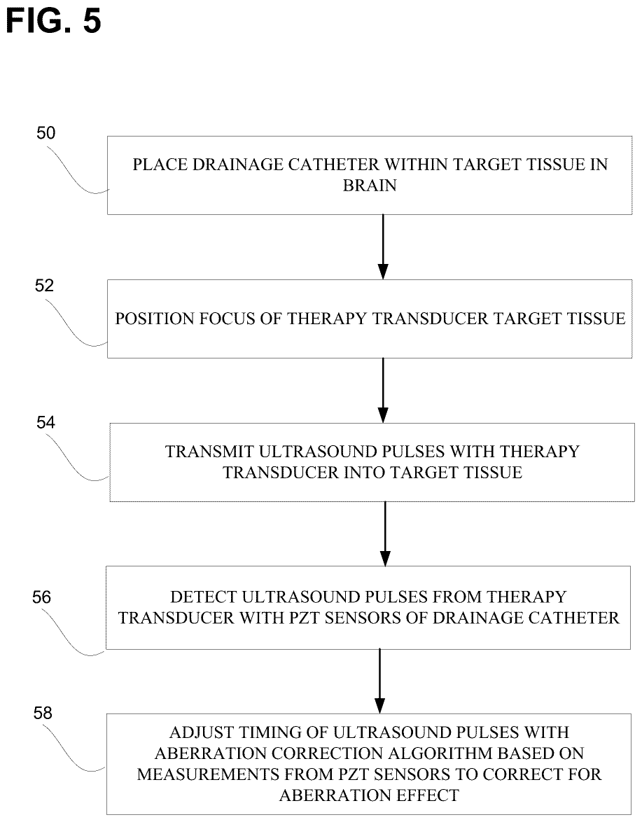

FIGS. 4-5 illustrate one embodiment and method for treating brain tissue with Histotripsy ultrasound therapy. FIG. 4 illustrates a therapy transducer 114 positioned adjacent to a skullcap of a patient, and a drainage catheter 117 positioned partially inside the brain of the patient such that drainage ports of the catheter are positioned within or adjacent to a target tissue of the brain, such as a blood clot or a brain tumor. To obtain precise focusing and focal steering, the PZT sensors in the catheter can be placed close to the geometrical focus of the transducer. In one embodiment, the therapy transducer 114 can include a hole 123 to facilitate catheter insertion through the transducer array. A catheter holder can be screwed into the hole, with scale markings on the catheter, which allows the operator to know the precise position of the catheter tip based on the insertion position, angle, and distance.

First, referring to step 50 of FIG. 5 and also to FIG. 4, the drainage catheter 117 can be inserted through the skullcap of the patient and placed within or adjacent to the target tissue in the brain. Next, referring to step 52 of FIG. 5 and also to FIG. 4, a focus of the therapy transducer 114 can be positioned on the target tissue. The therapy transducer itself can be acoustically coupled to the skull of the patient. Next, referring to step 54 of FIG. 5, ultrasound pulses can be transmitted from the therapy transducer into the target tissue. At step 56 of FIG. 5, PZT sensors of the drainage catheter can detect or measure ultrasound pulses from the therapy transducer. Finally, at step 58 of FIG. 5, the software and electronic controller of the system can adjust timing of the ultrasound pulses with an aberration correction algorithm based on the measurements from the PZT sensors to correct for the aberration effect caused by the skullcap.

The ultrasound pulses can be configured to generate cavitation or bubble clouds within the target tissue of the brain to liquefy the target tissue. In some embodiments, the liquefied target tissue can be drained with the catheter. In further embodiments, the focus of the therapy transducer can be electronically or mechanically steered to fully liquefy the target tissue.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. (canceled)

2. A method of providing histotripsy therapy to a brain of a human patient, comprising the steps of:
positioning a plurality of transducer elements of a therapy transducer outside a skullcap of the patient;
positioning a focus of the therapy transducer at a first treatment location within a target treatment volume of the brain;
transmitting histotripsy pulses from the plurality of transducer elements into the first treatment location of the target treatment volume through the skullcap to form a non-thermal bubble cloud in the target treatment volume;
identifying a time of flight of the histotripsy pulses from the transducer elements to the target treatment volume;
adjusting transmission of subsequent histotripsy pulses from the plurality of transducer elements to the first treatment location with an aberration correction algorithm based on the time of flight to automatically correct for an aberration effect caused by the histotripsy pulses passing through the skullcap;
electronically steering the focus of the therapy transducer to a subsequent treatment location within the target treatment volume; and
repeating the transmitting, identifying, and adjusting steps for the subsequent treatment location.

3. The method of claim 2, further comprising repeating the electronically steering, transmitting, identifying, and adjusting steps for all subsequent treatment locations within the target treatment volume until the target treatment volume is liquefied.

4. The method of claim 2, further comprising positioning a drainage catheter in the target treatment volume of the brain.

5. The method of claim 4, further comprising measuring the histotripsy pulses with a piezoelectric sensor disposed on a guidewire portion of the drainage catheter.

6. The method of claim 2, wherein forming the non-thermal bubble cloud in the target tissue volume liquefies the target tissue volume.

7. The method of claim 6, further comprising draining the liquefied target tissue volume from the brain with a sheath portion of the drainage catheter.

8. The method of claim 2, wherein the target region comprises a clot or hemorrhage.

9. The method of claim 2, wherein the target region comprises a brain tumor.

10. The method of claim 4, wherein positioning the drainage catheter further comprises positioning the drainage catheter through a hole of the therapy transducer.
